(19) United States
(12) Patent Application Publication
(10) Pub. No.: US 2026/0191675 A1
(43) Pub. Date: Jul. 9, 2026

(54) Post-Operative Compression Garment

(71) Applicant: MEDICAL Z, SAINT AVERTIN (FR)

(72) Inventors: Yann TEDGUI ZAGAME, Houston, TX (US); Alain Ali MOJALLAL, Lyon (FR)

(21) Appl. No.: 19/129,534

(22) PCT Filed: Nov. 15, 2022
(86) PCT No.: PCT/FR2022/000111

§ 371 Date: Jun. 23, 2025

(51) Int. Cl. A61F 5/03

(52) U.S. Cl. CPC A61F 5/03

(57) ABSTRACT

The invention relates to a post-operative compression garment designed to compress a part of a patient's body, characterised in that it comprises: at least one compression textile and a plurality of silicone tracks (1) arranged on a plurality of support strips arranged on a face of the compression textile intended to be in contact with the skin of the patient, the support strips having a common point in an upper portion of the garment, the distance between the support strips increasing at an increasing distance from the common point so that the silicone tracks adhere to the skin of the patient along a respective lymphatic drainage path of said part of the patient's body.

### FIELD OF THE INVENTION

The present invention relates to a post-operative compression garment.

### STATE OF THE ART

Following surgery, inflammation and the formation of edema and hematomas are very common. These edemas and hematomas are painful and can hinder the proper healing of the tissue. There is also a risk of blood clots forming. In some cases, these phenomena can take up to several weeks to disappear completely.

Applying compression to the area of edema and hematoma can reduce these side effects. However, applying compression to a patient's body is difficult to implement, particularly because each operation involves different detachments, dissections, infiltrations, edema and scars and requires appropriate compression, while protecting specific scar areas.

After plastic surgery, there may also be a risk of skin sagging, which could lead to unsightly results. This risk can be reduced by wearing a compression garment to hold the tissues in place and help with skin re-draping.

Compression needs evolve over time following surgery depending on the phases of healing and post-operative maturation. For example, some areas may be very fragile in the immediate post-operative phase, while compression treatment may be beneficial in a later phase.

### DISCLOSURE OF THE INVENTION

A purpose of the invention is to design a compression garment intended for post-operative use which provides improved healing of the treated anatomical area.

To this end, the invention provides a post-operative compression garment designed to compress a part of a patient's body, characterized in that it comprises:
at least one compression textile, and
a plurality of silicone tracks arranged on a plurality of support strips arranged on a face of the compression textile intended to be in contact with the patient's skin, said support strips having a common point in an upper portion of the garment, the distance between the support strips increasing at an increasing distance from the common point so that the silicone tracks adhere to the patient's skin along a respective lymphatic drainage path of said part of the patient's body.

Such a compression garment allows for customization by area of the patient's body and by surgery type. The silicone tracks allow for self-drainage of the lymphatic paths. Furthermore, the garment allows for flexible design depending on the post-operative period.

Wearing such a compression garment helps reduce swelling, bruising, and pain in the patient, and accelerates and improves healing. It also improves the comfort of the patient and speeds up the return to physical activity after surgery. Furthermore, wearing a compression garment improves skin remodeling and the outcome of the operation.

Advantageously, the compression textile comprises at least two areas joined by elastic seams, the excess seam of which is arranged on the outside of the garment. Thus, different compression gradients can be applied depending on the type of surgery.

Preferably, the silicone tracks form a relief on the support strip. Advantageously, the silicone tracks are wavy, preferably sinusoidal.

Advantageously, each support strip comprises several parallel silicone tracks, preferably two parallel silicone tracks.

In some embodiments, the compression garment is in the form of an abdominal compression girdle.

In other embodiments, the compression garment is in the form of a lower limb compression girdle.

In other embodiments, the compression garment is in the form of a breast compression girdle.

Another object of the invention relates to an assembly comprising a compression garment and at least one self-adhesive plate capable of being inserted between said compression garment and an area of the patient's body.

Another object of the invention relates to an assembly comprising a compression garment according to one of the preceding claims and at least one foam compression pad capable of being inserted between said compression garment and an area of the patient's body.

The invention also relates to a method for manufacturing a compression garment for a part of a patient's body, comprising
providing at least two support strips including at least one respective silicone track, and
arranging said at least two support strips on an inner face of a garment made of a compression textile, such that the silicone tracks adhere to the patient's skin, the support strips having a common point in an upper portion of the garment, the distance between the support strips increasing at an increasing distance from the common point along at least one lymphatic drainage path of said part of the patient's body.

### BRIEF DESCRIPTION OF THE FIGURES

Other characteristics and advantages of the invention will emerge from the detailed description which follows, with reference to the appended drawings, in which:

FIG. 1A is a schematic view of an abdominal compression girdle intended to be worn after abdominal liposuction.

FIG. 1B is a schematic view of an abdominal compression girdle intended to be worn after abdominal dermolipectomy with liposuction with low incision and central detachment.

FIG. 1C is a side view of an abdominal girdle worn by a patient.

FIG. 1D is a back view of an abdominal girdle worn by a patient.

FIG. 2A is a schematic view of the front face of a lower limb compression girdle.

FIG. 2B is a schematic back view of a lower limb compression girdle.

FIG. 2C is a side view of a lower limb compression girdle worn by a patient.

FIG. 2D is a back view of a lower limb compression girdle worn by a patient.

FIG. 2E illustrates a fabric area including support strips including silicone tracks, intended to form a portion of a lower limb compression girdle.

FIG. 3 is a schematic view of a breast compression garment intended to be worn following breast surgery.

FIG. 4 illustrates a silicone strip suitable for being arranged according to the anatomical lymphatic drainage paths.

FIG. 5 illustrates a self-adhesive plate designed to be used in conjunction with a compression garment.

FIG. 6 illustrates a foam compression pad designed to be used in conjunction with a compression garment.

### DETAILED DESCRIPTION OF EMBODIMENTS

The invention provides a compression garment intended to be worn typically after a plastic and cosmetic surgery procedure inducing edema and/or a scar in a part of a patient's body. However, the use of such a garment is not limited to plastic surgery but can also be considered after another type of surgical procedure. As an illustrative and non-limiting example, the garment may be intended to be worn after a digestive surgery procedure with median laparotomy, orthopedic surgery with knee or hip incisions and breast surgery.

The garment is designed according to the concerned anatomical area and according to the surgical procedure performed on the patient. For example, an abdominal compression girdle can be designed for abdominal liposuction alone without incision and without detachment or for lipo-abdominoplasty with low incision and central detachment, or for a vertical midline laparotomy. A lower limb girdle can be designed for liposuction of the lower limbs alone or a thigh dermolipectomy (cruroplasty) with liposuction with a midline or postero-median incision. A breast compression garment can be designed for breast reduction with mastopexy or for breast augmentation with prostheses alone or by composite technique including a prosthesis and autologous fat injection.

The compression garment comprises at least one textile area having an extensibility chosen according to the compression to be exerted on the part of the patient's body covered by said area. Each textile is typically made of a warp and weft fabric, which is stretchable in the warp and weft, that is to say that this fabric is able to lengthen elastically along its warp yarns and along its weft yarns.

Each warp and weft yarn may comprise in its composition: natural fibers, such as cotton or linen synthetic fibers, such as polyamide, polyester, nylon, elastane or Lycra™, or a mixture of several of the aforementioned fibers.

A textile of this type has a maximum elongation in the warp and weft, that is to say, an elongation at break in the direction of its warp and weft yarns. The fabric(s) constituting the compression garment typically have a substantially homogeneous stretch in the warp and weft. In other embodiments, other textile structures and other stretches may be used.

When the compression garment is worn by the patient, each textile area exerts compression on the patient's body, the intensity of which depends on the stretch of the textile. A high textile stretch results in low compression on the patient's body, and a low textile stretch produces high compression on the patient's body. The stretch of each textile can therefore be adjusted to the desired effect, namely to exert sufficient compression to facilitate or accelerate the reduction of post-operative edema and hematomas while exerting less pressure on the areas weakened by the surgical intervention, allowing the patient's movements. Generally, the compression is comprised between 17 and 24 mm Hg. Optionally, several fabric areas with different extensibilities can be joined in a garment.

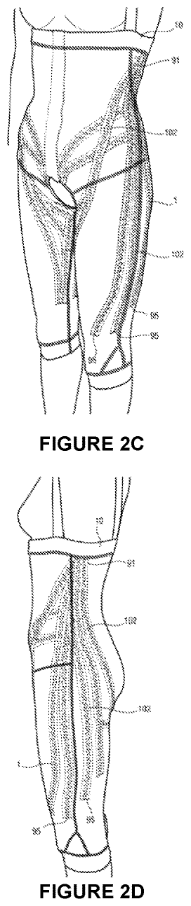

The compression garment moreover includes silicone tracks 1 arranged on the inner surface of the garment. Each silicone track is carried by an elastic textile support strip attached to the inside of the garment. Said silicone tracks 1 are oriented according to the anatomical lymphatic drainage paths, which are known to the person skilled in the art. The silicone tracks are for example arranged in a zigzag or sinusoidal pattern and may have a relief, for example with a height of 2 mm. In practice, one or more silicone tracks may be arranged on one face of a support strip in the form of a textile ribbon, the opposite face of which may be applied to the inner face of the garment and attached by sewing or gluing. FIG. 4 illustrates such a support strip in the form of a textile ribbon 102. The support strip comprises two embossed silicone tracks 101. Advantageously, the tracks are corrugated, preferably sinusoidal. When the garment is worn, the silicone tracks 1 are applied against the patient's skin. The silicone provides a certain adhesion of the silicone tracks 1 to the skin. Thus, when the patient moves, the micro-movement and micro-shearing effect generated by these silicone tracks adhering to the skin acts as guided lymphatic drainage directing the lymph to the lymph node areas. In addition, thanks to these silicone tracks adhering to the skin, the compression garment is better stabilized on the skin, particularly in areas that are difficult to hold, such as, for example, in the case of an abdominal compression girdle, the roots of the thighs or the subcostal region.

In a particularly advantageous embodiment, several silicone tracks are arranged together on a support strip which is held on the fabric by one or more seams. Preferably, two parallel silicone tracks are in contact with the patient's skin when the garment is worn. The two tracks have a raised sinusoidal pattern. The presence of two parallel tracks allows for improved retention of the garment on the skin of the garment compared to the use of a single track.

The arrangement of the silicone tracks and the support strips is such that several silicone tracks meet at a common point in an upper area of the garment. In the case of trousers or a girdle, the common point is located at the patient's waist. The support strips including the silicone tracks radiate downward from this common point along the patient's lymphatic paths, so that the distance between the respective support strips increases with increasing distance from the common point, towards the bottom of the garment.

Advantageously, the textile areas constituting the compression garment are joined together by elastic seams, the excess seam of which is preferably arranged on the outside of the garment. Said elastic seams are preferably arranged so as not to cover the incision area and the area of detachment from the patient's body when the garment is worn by the patient.

Different embodiments of such a compression garment will now be described.

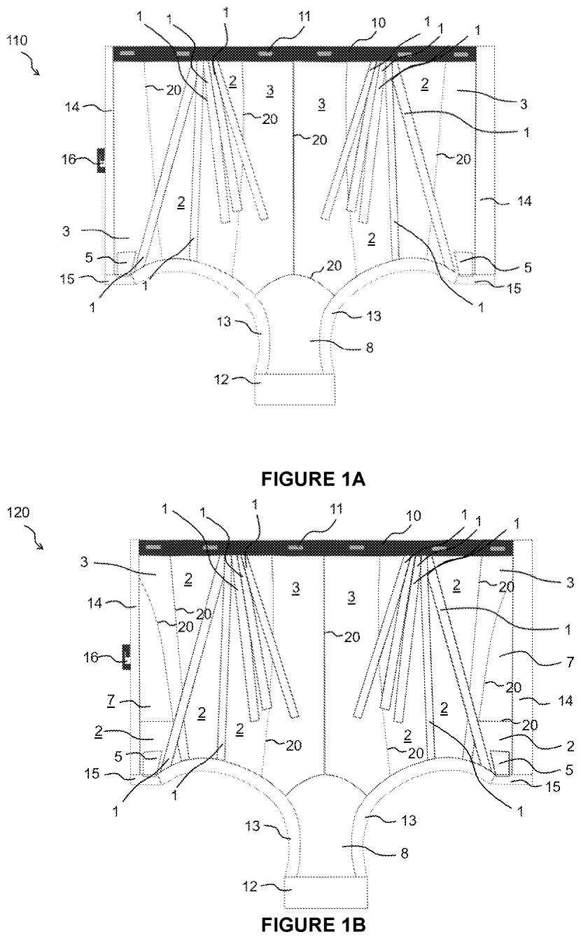

FIG. 1A is a schematic view of a first embodiment of a post-operative compression garment, namely a first abdominal compression girdle 110. The girdle 110 is designed to be worn by a patient after a surgical procedure on the patient's abdomen, and in particular after an abdominal liposuction procedure.

The abdominal compression girdle 110 includes several areas of a first textile 2 and several areas of a second textile 3. The first textile 2 has an extensibility of less than that of the second textile 3. The different textile areas are advantageously joined by elastic seams 20. Moreover, the girdle comprises several silicone strips 1 arranged along lymphatic drainage paths of the abdominal region.

When the girdle is worn by the patient, a first area including the first textile 2 is arranged so as to exert a first compression imposing a concave shape in the posterior and lateral regions of the patient, and the second area including the second textile 3 is arranged so as to exert a second homogeneous compression less than the first compression exerted on an abdominal region of the patient.

In combination with these compressions, the silicone tracks 1 ensure lymphatic drainage of the abdominal region.

On the upper edge, the girdle 110 includes an elastic band 10 comprising loops 11 capable of attaching suspenders (not shown). Furthermore, the girdle 110 includes a door strip 14 allowing the girdle to be closed at the patient's stomach by hooks 16. An elastic bias 15 is arranged at the lower edge of the closing area.

Said girdle 110 includes a cotton base 8 capable of being closed and opened by a soft Velcro strip 12 which is designed to be hooked to the gripping Velcro strips 5, said base 8 allowing to hold the girdle in place when it is worn by the patient. Lace strips 13 can be arranged on the openings allowing the passage of the thighs.

FIG. 1B shows a second embodiment of a post-operative compression garment, namely a second abdominal compression girdle 120.

The elements designated by the same reference signs as in FIG. 1A are identical or fulfill the same function as the elements already described in the first embodiment and will therefore not be described again.

The girdle 120 is designed to be worn by a patient following surgery on the patient's abdomen, and in particular following an abdominoplasty procedure. such as abdominal dermolipectomy with liposuction with low incision and central detachment.

The abdominal compression girdle 120 includes several areas of a first textile 2, several areas of a second textile 3 and one or more areas of a third textile 7. The first textile 2 has an extensibility of less than that of the second textile 3, which is itself less than that of the third textile 7. The different textile areas are advantageously joined by elastic seams. A first area including the first textile 2 is arranged so as to exert a first compression imposing a concave shape in posterior and lateral regions of the patient, and the second area including the second textile 3 is arranged so as to exert a second homogeneous compression and less than the first compression exerted on a part of the abdominal region of the patient, and the third area comprising the textile 7 exerts a very low pressure on the anterior and central area which corresponds to the detachment area.

FIGS. 1C and 1D show an abdominal compression girdle 120 worn by a patient. The support strips 102 having the silicone tracks 1 indicated in dotted lines are arranged on the inner face of the girdle in contact with the patient's skin.

The support strips including silicone tracks meet at a common point 90 near the upper edge of the girdle including an elastic band 10. The distance between the support strips including silicone tracks increases towards the lower part of the girdle and is maximum at the lower edge having an elastic bias 15.

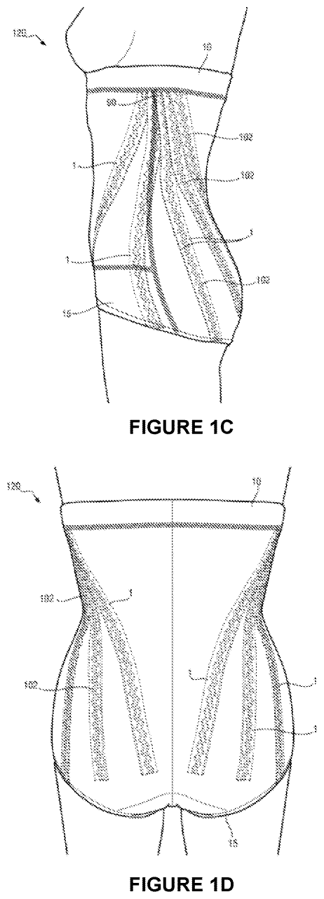

FIGS. 2A and 2B show a lower limb compression girdle 210 designed to be worn after liposuction of the thighs or buttocks. FIG. 2A shows a schematic view of the front face of said girdle 210, FIG. 2B shows a schematic view of the back view of said girdle 210.

The lower limb compression girdle 210 includes several areas of a textile 22 joined together by elastic seams 20. Moreover, the girdle comprises several silicone tracks 1 arranged along lymphatic drainage paths of the lower limbs. On the upper edge, the lower limb compression girdle 210 includes an elastic band 10 comprising loops 11 capable of attaching suspenders (not shown). Furthermore, the girdle 210 includes two door strips 14 which allow the pants to be closed on the sides by hooks 16.

Said girdle 210 may further include an opening at the pubis (not shown) or, alternatively, include a closed bottom.

The girdle 210 may further include an elastic bias 25 crossing the width of the front face of the pants arranged at the pubis, arranged to form a border for the portion of the fabric constituting the edge of the opening on the front face if necessary. Lace strips 13 may be arranged on the lower openings allowing the passage of the feet. At the calves, pieces 23 of a fabric having a high stretch may be arranged, exerting very low pressure and thus increasing the comfort of the patient wearing the girdle 210 of the lower limbs.

FIGS. 2C and 2D show a lower limb compression girdle worn by a patient. The support strips 102 having the silicone tracks 1 indicated in dotted lines are arranged on the inner face of the girdle in contact with the patient's skin.

The silicone tracks meet at a common point 91 near the upper edge of the girdle including an elastic band 10. The distance between the silicone tracks increases towards the lower part of the girdle and is maximum at their lower end 95 at the patient's knee.

FIG. 2E illustrates an area of fabric cut to form a portion of the lower limb compression garment. The support strips including silicone tracks are sewn onto the area of fabric. Several support strips having silicone tracks join at a common point intended to form the upper edge of the garment to which an elastic band will be attached. Other support strips having silicone tracks to apply compression to other lymphatic drainage paths are arranged on the fabric area.

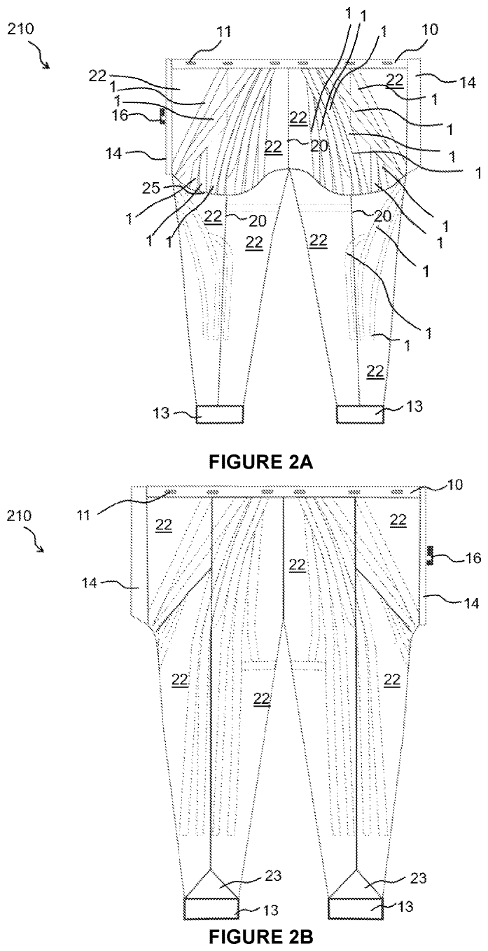

FIG. 3 shows a breast compression garment 310 designed to be worn after breast surgery. Such surgery is, for example, a breast reduction with mastopexy or a breast augmentation, for example, with prostheses alone or with a composite technique comprising a prosthesis and an injection of autologous fat.

The breast compression garment 310 includes several areas of a first textile 2 and several areas of a second textile 3. The first textile 2 has an extensibility of less than that of the second textile 3. Moreover, the garment 310 comprises several support strips comprising silicone tracks 1 arranged along lymphatic drainage paths of each breast.

When the garment 310 is worn by the patient, a first area comprising the first textile 2 is arranged so as to exert a first compression between the lateral part of the breast and the latero-thoracic part, allowing shaping of the breast from the latero-mammary fold. The second area including the second textile 3 is arranged so as to exert a second compression less than the first compression on the lateral parts and the central part of the back. In combination with these compressions, the silicone tracks 1 ensure lymphatic drainage of the breasts.

In a central area of the back and on a part of each breast, the garment further includes a thermoregulatory lining, arranged below an area of the second textile 3. Said thermoregulatory lining allows to reduce the patient's perspiration.

The breast compression garment includes an elastic waistband 38 arranged below the breast level, capable of holding the garment in place. A divisible zipper 35 and hooks 36 are arranged on the front edges of the garment, allowing closure in a central area of the patient's chest. The garment may include a foam piece 33 in the central area of the chest, increasing the comfort of the patient. Furthermore, one or more ribbons 34 may be arranged in said area forming a border on the front edges of the garment.

Two bands 37 are arranged in the part above the breast and passing over the patient's shoulders, said bands 37 each comprising several fasteners 39 capable of being attached on loops 49 arranged on both sides of the central part of the back of the garment, said several fasteners allowing to adapt the length of the band 37 necessary for supporting the breast by choosing the fastener 39 most suited to the morphology of the patient.

The invention is not limited to the garment examples described but is applicable to a multitude of post-operative compression garments with lymphatic drainage, by applying to the latter the technical considerations developed so far. Other embodiments may comprise, in an illustrative and non-limiting manner, compression garments covering the arms, the back, or a single upper or lower limb.

The compression garment can be used in conjunction with other items that may be added in later phases of healing following surgery.

For example, in the early phase of healing, it may be preferable not to exert too much compression on the operated tissues.

When the surgical intervention has generated a scar, it may be useful, in a second phase in which the scar is closed, to apply a suitable element to the scar to massage the scar in order to fade it.

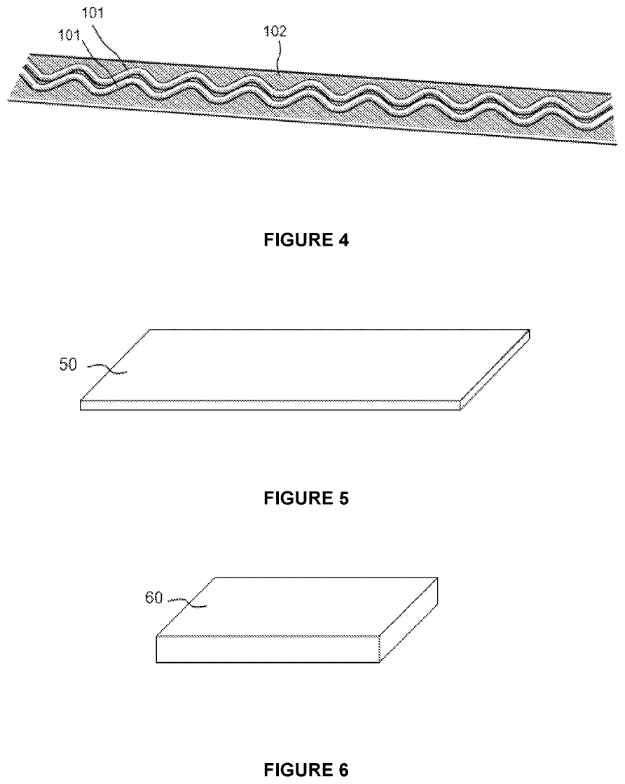

Moreover, one or more foam compression pads 60, as illustrated in FIG. 6, may be arranged between the compression garment and the patient's body, to increase compression in an initially slightly compressed area. For example, such pads may be placed in the anterior and sub-umbilical portion which corresponds to the detachment area when the garment is an abdominal compression girdle. Such foam compression pads 60 are typically added approximately 5-7 days after surgery. Compression before this time may reduce perfusion and increase the risk of skin necrosis. The purpose of the pads is to compress strongly to shape and flatten the skin in order to improve the aesthetic result of the surgery. Although the pad 60 is shown with a parallelepiped shape in FIG. 6, it goes without saying that any other shape, in particular one adapted to the patient's morphology, may be used.

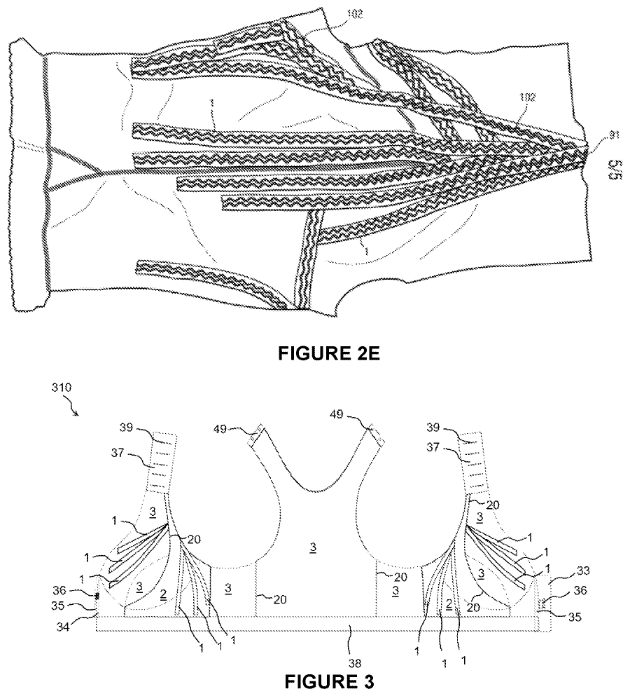

Moreover, in certain situations, one or more self-adhesive plates 50, as illustrated in FIG. 5, may be inserted between the compression garment and the patient's body, at the healing scar. For example, such plates 50 may be placed at the subpubic fold when the garment is an abdominal compression girdle used after an abdominoplasty. These plates 50 are first adhered to the patient's skin then are held in place by the pressure applied by said compression garment. Such plates 50 are typically inserted after the incision has healed, which typically lasts between 3 and 4 weeks. Said plates, which are conformable to the patient's body, may be made of silicone or a mixture of tri-block copolymer and mineral oil. Although the plate 50 is shown with a parallelepiped shape in FIG. 5, it goes without saying that any other shape, particularly one adapted to the patient's morphology, can be used.

1. A post-operative compression garment designed to compress a part of a patient's body, said post-operative compression garment comprising:
at least one compression textile, and
a plurality of silicone tracks arranged on a plurality of support strips arranged on a face of the compression textile intended to be in contact with the patient's skin, said support strips having a common point in an upper portion of the garment, a distance between the support strips increasing at an increasing distance from the common point so that the silicone tracks adhere to the patient's skin along a respective lymphatic drainage path of said part of the patient's body.

2. The post-operative compression garment according to claim 1 wherein the at least one compression textile comprises at least two areas joined by elastic seams, an excess seam of which is arranged on the outside of the post-operative compression garment.

3. The post-operative compression garment according to claim 1, wherein the silicone tracks form a relief on the support strip.

4. The post-operative compression garment according to claim 1, wherein the silicone tracks are wavy, preferably sinusoidal.

5. The post-operative compression garment according to claim 1, wherein each support strip comprises several parallel silicone tracks, preferably two parallel silicone tracks.

6. The post-operative compression garment according to claim 1, in the form of an abdominal compression girdle.

7. The post-operative compression garment according to claim 1, in the form of a lower limb compression girdle.

8. The post-operative compression garment according to claim 1, in the form of a breast compression girdle.

9. An assembly comprising a post-operative compression garment according to claim 1, and at least one self-adhesive plate capable of being inserted between said post-operative compression garment and an area of the patient's body.

10. The assembly comprising a post-operative compression garment according to claim 1, and at least one foam compression pad capable of being inserted between said post-operative compression garment and an area of the patient's body.

11. A method for manufacturing a post-operative compression garment for a part of a patient's body, comprising:
providing at least two support strips including at least one respective silicone track, and
arranging said at least two support strips on an inner face of a garment made of a compression textile, such that the silicone tracks are oriented towards the patient's skin side, the support strips having a common point in an upper portion of the garment, the distance between the support strips increasing at an increasing distance from the common point along at least one lymphatic drainage path of said part of the patient's body.
